(19) United States
(12) Patent Application Publication
(10) Pub. No.: US 2003/0170828 A1
(43) Pub. Date: Sep. 11, 2003

(54) Synthesis of complex carbohydrates

(76) Inventors: Wei Zou, Gloucester (CA); Harold J Jennings, Gloucester (CA)

Correspondence Address: W Charles Kent, Ridout & Maybee, 150 Metcalfe Street 19th Floor, Ottawa, ON, K2P 1P1, CA

(21) Appl. No.: 10/344,958

(22) PCT Filed: Aug. 22, 2001
(86) PCT No.: PCT/CA01/01208

(30) Foreign Application Priority Data
Aug. 22, 2000 (US) 60226980

(51) Int. Cl. C12P 19/26; C12P 19/04; C08B 37/00

(52) U.S. Cl. 435/84; 435/101; 536/53

(57) ABSTRACT

A tailor-assembly approach is employed for synthesis of complex carbohydrates wherein a polysaccharide is degraded and the shorter product obtained from the degradation is subjected to enzymatic modification to add a sugar moiety. The products may be useful in the preparation of a cancer vaccine. In one example, oligosaccharides of the type Ia group B Streptococcus (GBSIa) capsular polysaccharide and multivalent sialyl Le^{x} antigens are specifically described. GBSIa polysaccharide was depolymerized by partial Smith degradation to fragments representing asialo core repeating units. Enzymatic sialylation of these oligomers furnished GBSIa repeating units (from monomer to pentamer). Fucosylation on GlcNAc residues of GBSIa oligomers afforded oligosaccharides that carry multiple sialyl Le^{x} epitopes.

### FIELD OF THE INVENTION

The invention relates to the field of synthesis of complex carbohydrates.

### BACKGROUND OF THE INVENTION

The development of carbohydrate-based anticancer and antibacterial vaccines has lead to a need for more efficient methods for the synthesis of complex carbohydrate antigens. Although notable progress has been made in chemical and chemo-enzymatic synthesis (e.g. Zou, Carbohydr.Res, 1998, 309,297), solid-phase synthesis (e.g. Zheng, Angew.Chem., (Int. Ed. Engl.), 1998, 37, 786-788) and programmed robotic synthesis (e.g. Zhang, J.Am.Chem.Soc., 1999, 121, 734.), complex carbohydrates of biological significance are still very difficult to make. This difficulty is compounded by the fact that most biological interactions between carbohydrates and proteins are multivalent, thus requiring for maximum efficiency the synthesis and presentation of multiple carbohydrate epitopes with defined structures.

Multivalent carbohydrate epitopes can be chemically or chemo-enzymatically synthesised. However, the methods widely used for the synthesis of oligosaccharides are time consuming, difficult, and expensive. Current methods share a common strategy wherein the oligosaccharide is built up step by step from monosaccharides and/or other small building blocks. The multiple steps involved in obtaining various monovalent carbohydrate epitopes limits their efficient synthesis, and obtaining them in a multivalent form adds a further degree of difficulty.

Thus, it is an object of the present invention to provide an improved method for the synthesis of complex carbohydrates.

### SUMMARY OF THE INVENTION

There is provided a novel and surprising method for the synthesis of complex carbohydrates including multivalent carbohydrate antigens by employing “a tailor-assembly approach.” This approach involves controlled degradation of a polysaccharide to form a shorter product followed by enzymatic addition of sugar moieties to the shorter product to make the desired complex carbohydrate.

Prior methods have been limited to complete chemical or chemo enzymatic synthesis wherein mono or disaccharides form the basis to which numerous sugar moieties must be added in a painstaking stepwise fashion. Prior attempts to use polysaccharide degradation products as building blocks in synthesis have proven unsatisfactory as the degradation products have been mono or disaccharides which are not suitable for use in the production of multivalent carbohydrate antigens.

In contrast, the present invention comprises the novel combination of controlled degradation with construction methods from the field of enzymology to overcome limitations of prior methods. The controlled degradation permits the production of oligosaccharides suitable for enzymatic modification to produce multivalent carbohydrate antigens.

The method disclosed herein may be used to prepare complex carbohydrates suitable for use as antigens, including multivalent antigens.

In an embodiment of the invention there is provided a method of synthesizing complex carbohydrates comprising: (a) subjecting a polysaccharide to degradation to produce a shorter product having at least 4 saccharides; and, (b) subjecting the shorter product to an enzyme-mediated process wherein a first sugar moiety and a second sugar moiety are linked to a first and a second site respectively on the shorter product by an O-glycosidic bond to produce multiple potential antigenic epitopes.

In an embodiment of the invention there are provided multivalent carbohydrate antigens.

### BRIEF DESCRIPTION OF THE DRAWINGS

FIG. 1 is a depiction of the structure of two repeating units of GBSIa capsular polysaccharide.

FIG. 2 is a depiction of the structure of two repeating units of GBSIb capsular polysaccharide.

FIG. 3 is a depiction of the structure of an embodiment of a multivalent sialyl Lewis-x antigen.

FIG. 4 is a depiction of the structure of an embodiment of a multivalent Lewis-x antigen.

FIG. 5 is a depiction of the structure of an embodiment of a multivalent Lewis-y antigen.

FIG. 6 is a depiction of the structure of an embodiment of a multivalent sialyl Lewis-a antigen.

FIG. 7 is a depiction of the structure of an embodiment of a multivalent Lewis-a antigen.

FIG. 8 is a depiction of the structure of an embodiment of a multivalent Lewis-b antigen.

FIG. 9 is a schematic presentation of an embodiment of the application of the tailor-assembly approach to the synthesis of multivalent Lewis antigens.

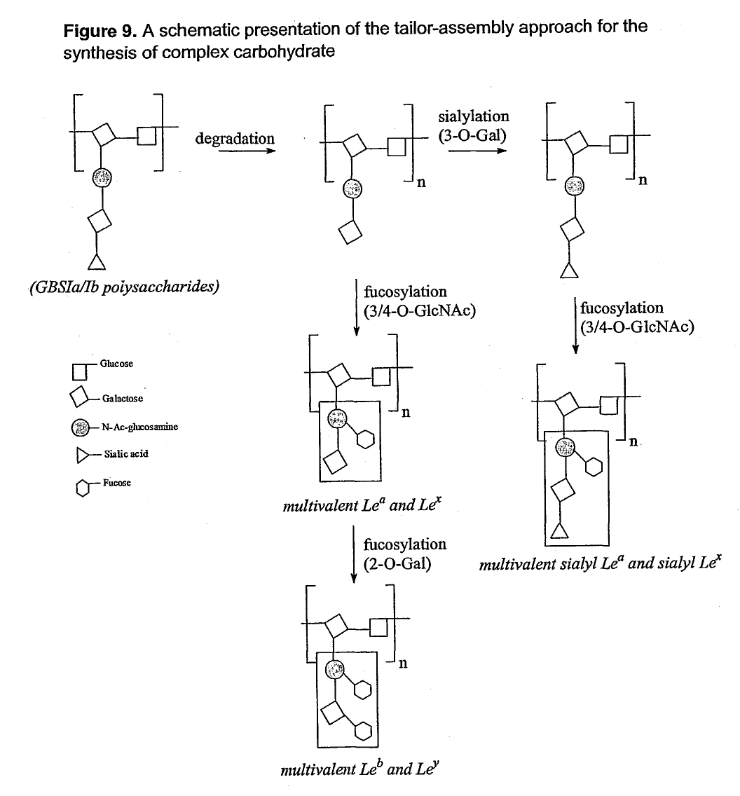

FIG. 10 is a representation of an embodiment of the procedure for synthesis of multivalent sialyl Lewis-x from GBSIa.

FIG. 11 is a graphical depiction of the ¹H NMR spectra of an embodiment of a trivalent sialyl Lewis-x antigen.

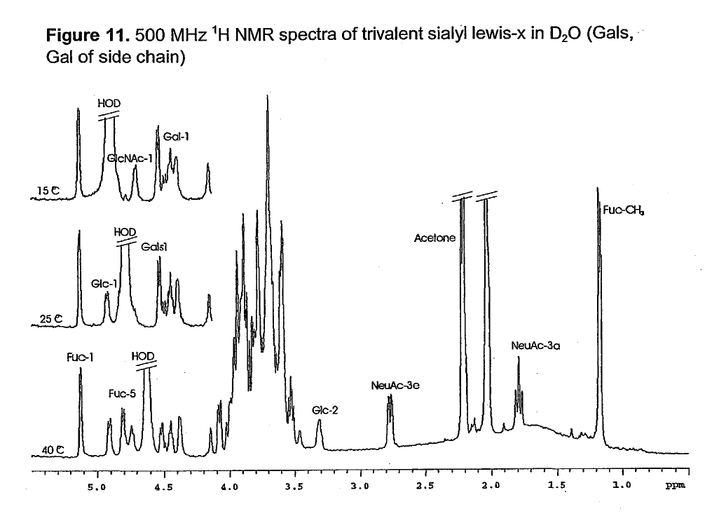

FIG. 12 is a depiction of oligosaccharides resulting from the embodiment of the degradation described in Example 2.

FIG. 13 is a depiction of products of an embodiment of the fucosylation described in Example 4.

### DETAILED DESCRIPTION OF THE PREFERRED EMBODIMENTS

A schematic illustration of an embodiment of the method of the invention follows:

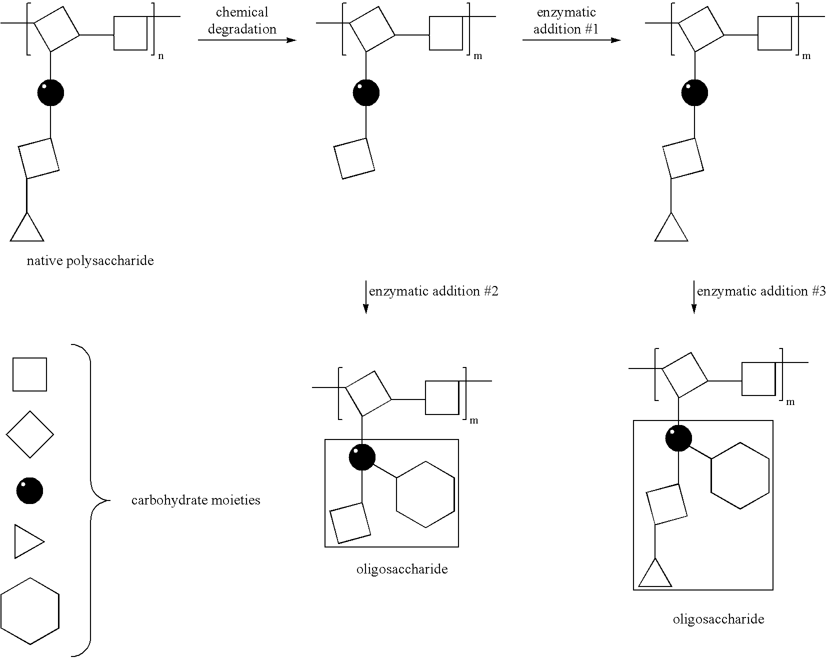

Where the production of a multivalent antigen is desired, “m” as depicted above is preferably between 2 and 10, more preferably between 4 and 8. “m” may be any multiple of 0.5 greater than 1. “m” is less than “n”. “n” may be very large and is limited only by the size of available naturally occurring polysaccharides.

Various carbohydrate moieties may be enzymatically added. The symbols used to represent carbohydrate moieties in the diagram above are not restricted by the legends of subsequent figures, and different symbols may represent the same carbohydrate moiety. The present invention is particularly useful for the generation of multivalent antigens. As sugar moieties in side chains may be principle immunodeterminants, the method disclosed herein is well suited for the design of multivalent antigens having desired characteristics.

As used herein, the term “multivalent antigen” refers to a complex carbohydrate having a backbone containing sugar moieties linked by 1,4-O-glycosidic bonds and having at least two branch (or “side chain”) sugar moieties, each of which is linked to at least one sugar moiety in the backbone. The term “multivalent antigen” as used herein is not limited to compounds for which antigenic properties are shown, and includes potential antigenic compounds.

As used herein, the term “complex carbohydrate” refers to carbohydrate polymers containing at least 10 sugars linked by O-glycosidic bonds. As used herein, the term “polysaccharide” refers to carbohydrate polymers having at least 40 sugars linked by O-glycosidic bonds.

Preferred polysaccharides for the method disclosed herein are polysaccharides having a backbone comprising known repeating units linked to known side chains. In some instances, preferred polysaccharides are Group B Streptococcus (“GBS”) polysaccharides.

It is desirable to know the structure of the polysaccharide to be degraded to permit efficient post-degradation modifications and generation of an intended antigen. Nonetheless, the method may be applied to polysaccharides of unknown sequence. However, in such cases it will not be possible to predict the identity of reaction products in advance.

As used herein, the term “shorter product” refers to a product of the controlled degradation of a polysaccharide, wherein the product has at least 3 saccharides linked by 1,4-O-glycosidic bonds.

While the invention is described with reference to particular examples, it will be readily understood that a variety of applications are contemplated and supported by the disclosure herein.

For example and not by way of limitation, while Smith degradation of polysaccharides (a combination of oxidation-reduction-acid hydrolysis, conventionally conducted using sodium periodate) is described, a variety of other methods of controlled polysaccharide degradation may be substituted. For example, a variant of the Smith degradation in which lead tetraacetate (Pb(OAc)₄) is used as an oxidant is also suitable, as are ozonolysis (e.g. U.S. Pat. No. 6,027,733) and other oxidation-reduction treatments (eg. U.S. Pat. No. 6,045,805) which are known in the art. The disclosure herein teaches a novel application of these degradation methods wherein they are used to degrade polysaccharides to produce “shorter products” of a size large enough to provide a backbone structure to which new or additional side chain sugar moieties can be added. The size of products of polysaccharide degradation can be modulated by variation of reaction conditions, such as reagent concentration and treatment duration. It is within the capacity of one skilled in the art, in light of the disclosure herein, to identify suitable degradation methods and apply them in a controlled manner to obtain useful shorter products.

Thus, the method disclosed herein permits existing polysaccharides having a suitable structure to be “tailored” into complex carbohydrates of interest by degradation and enzymatic addition.

Commonly, the shorter product is further modified by fucosylation or sialylation. However, various sugar moieties may be added to the partially degraded polysaccharide (“shorter product”) either instead of, or in addition to fucosylation or sialylation (e.g. U.S. Pat. No. 5,288,637). Different complex carbohydrates may be produced by appropriate adjustment of the degradation and addition stages. For example, Lewis antigen analogues bearing a truncated sialic acid moiety (NeuAc C-7, lacking the C8-9 exocyclic chain) may be generated by Smith degradation without a complete desialylation step. When a substantially homogeneous population of Lewis or sialyl Lewis antigens not including NeuAc C-7 is desired, Smith degradation may be followed by a separate desialylation step, to remove NeuAc C-7. Other sugars of interest for addition to the shorter product include: L-fucose, L-galactose, D-galactose, D-glucose, N-acetyl-D-glucosamine, and N-acetyl-D-galactosamine.

Similarly, sugar moieties to be added may be modified according to a variety of methods known in the art. For example, sialic acid may be modified by N-acylation, wherein the acyl group is preferably propionyl, butyril or benzoyl.

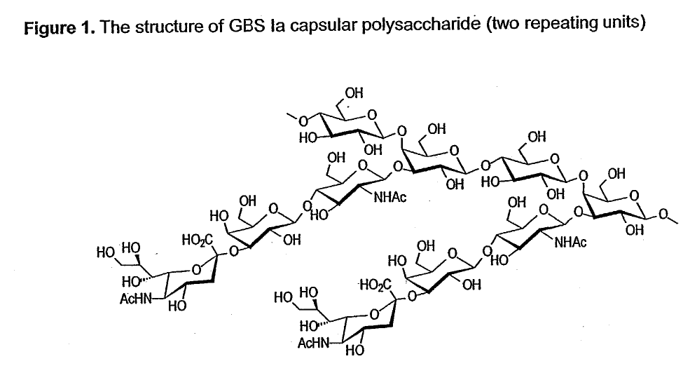

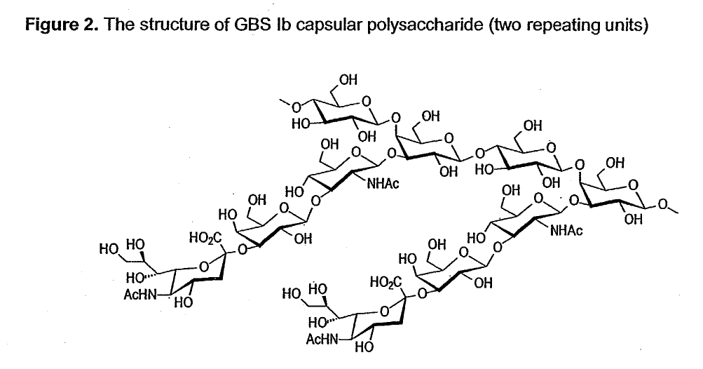

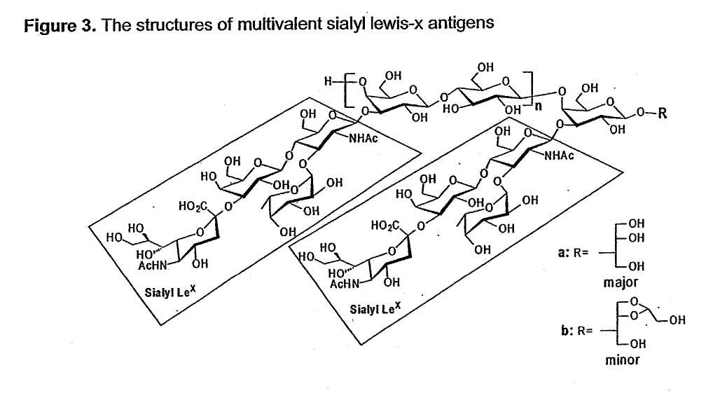

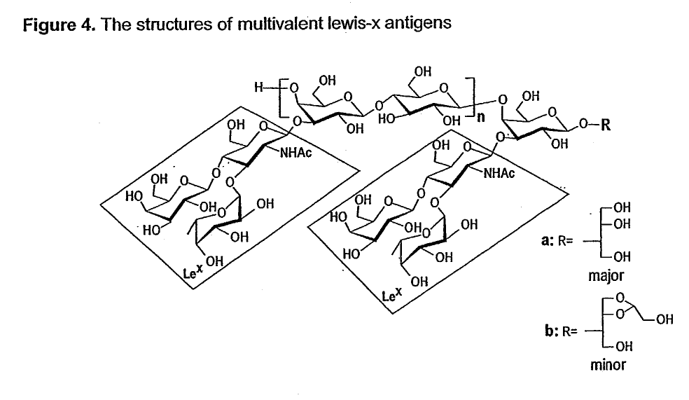

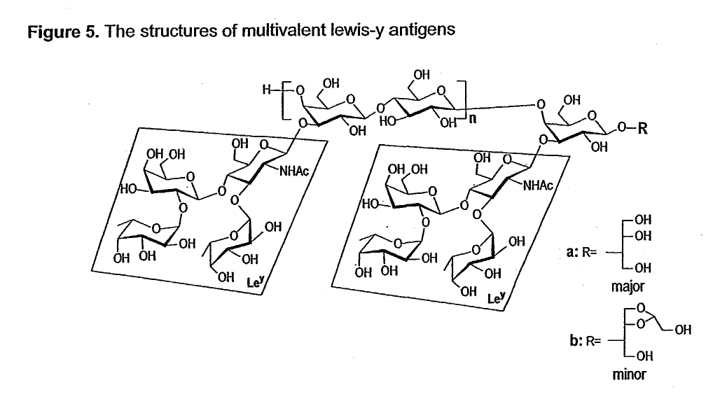

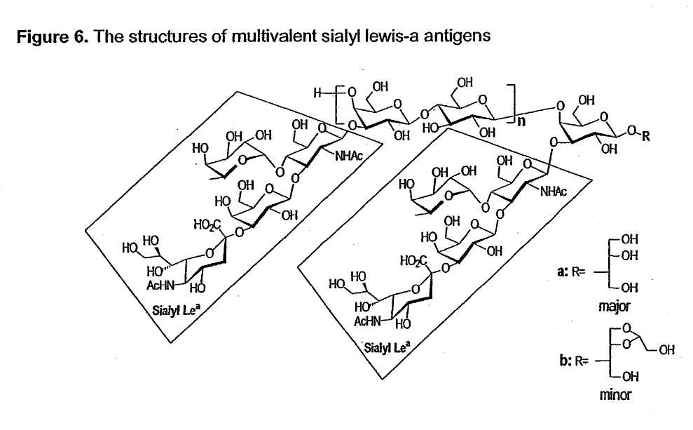

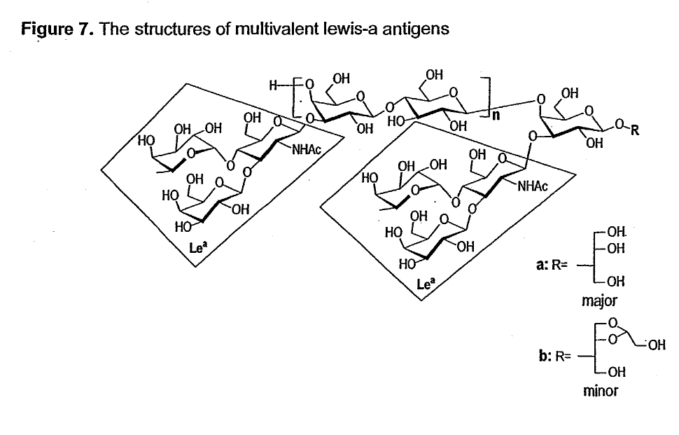

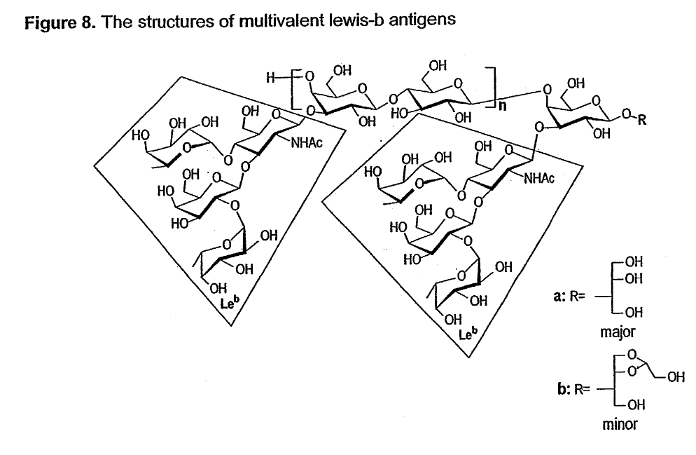

A broad range of complex carbohydrates can be prepared using the method herein disclosed. One useful group of complex carbohydrates are known as “Lewis antigens” (“Le”), including Lewis-y (Kim, Cancer Res., 1996, 46, 5985), (Leon, Int. J. Cancer, 1992, 51, 225), Lewis-b (Sakamoto, Cancer Res., 1986, 46, 1553), and sialyl Lewis-x and Lewis-a (e.g. Irimura, Adv. Exp. Med. Biol., 1994, 353, 27). While synthesis of particular antigens is discussed herein, it will be understood that in some instances the combination of several identical or different antigens in a single molecule will be desirable. For example, multivalent sialyl Lewis-x (FIG. 3) and -a (FIG. 6), Lewis-x (FIG. 4), Lewis-a (FIG. 7), Lewis-y (FIG. 5) and Lewis-b (FIG. 8) antigens from GBSIa (FIG. 1) or GBSIb (FIG. 2) are specifically described herein. While Lewis antigens are described as examples, the method is equally applicable to other complex carbohydrates.

Where Lewis antigens are produced, the carbohydrate backbone of the Lewis antigen is preferably between about 3 and 81 sugar moieties long, more preferably between about 5 and 21 sugar moieties long.

It will be readily appreciated that sugar moieties may be linked to any backbone residue of the shorter product. Thus, for example, sugar moieties may be linked to residues near one or the other end or toward the middle of the shorter product.

In some instances, at least one sugar moiety is linked to the product of polysaccharide degradation by a 1,3-O-glycosidic bond. In some instances, at least one sugar moiety is linked to the product of polysaccharide degradation by a 1,6-O-glycosidic linkage.

It will be appreciated that the initial degradation step will impact the number and type of side chain sugar moieties in the shorter product prior to enzymatic addition of sugar moieties. Thus, where a particular side chain composition or side chain location is desired, controlled degradation can be employed to preserve useful side chain features.

Similarly, the carbohydrate for degradation may be selected based on the type and/or composition of side chains it provides. Undesired side chain sugar moieties may be removed prior to the addition step using an appropriate glycosidase in aqueous buffer. Depending on the residues to be removed, suitable glycosidases include (but are not limited to) neurominidase, galacosidase, glucosidase, -acetyl-D-glucosidase and mannosidase. Removal of sialic acid is also contemplated as previously discussed. Thus, in light of the disclosure herein, it is within the capacity of one skilled in the art to identify suitable polysaccharides for degradation and to identify and use suitable glycosidases to remove unwanted side chain sugar moieties.

In light of the disclosure herein, it is within the capacity of a skilled technician to select and apply suitable degradation methods and construction enzymes to produce complex carbohydrates of interest.

General Methods. In the Examples provided below, ¹H and ¹³C NMR spectra were recorded at 500 MHz and 125 MHz, respectively, with INOVA-500 instrument at 293 K unless otherwise noted. Chemical shifts are given in ppm relative to the signal of internal acetone δ_{H} 2.225 in D₂O for ¹H NMR spectra, and to δ_{c} 31.07 for ¹³C NMR spectra. The ¹H NMR chemical shifts of oligosaccharides were assigned on the basis of 2D ¹H—COSY and ¹H—¹³C chemical-shift correlated experiments. Electron-spray mass spectroscopy (“ES-MS”) and capillary electrophoresis mass spectroscopy (“CE-MS”) were performed with QUATTRO (MICROMASS) and CRYSTALCE SYSTEM (trademark), respectively. MALDI-mass spectroscopy (“MS”) spectra were recorded with Voyager-DE™ STR (PerSeptive Biosystems).

Sialyl Le^{x/a} is a carbohydrate ligand for selectins and is believed to contribute to the hematogenous metastasis of cancer, and enhanced expression of sialyl Le^{x/a} a on epithelial mucins is correlated to the progression and poor prognosis of carcinomas. However, monovalent sialyl Lewis-x/Lewis-a (“Le^{x/a}”) binds with low affinity to the selectins, and recognition of mucin ligands by selectins requires the multivalent presentation of sialyl Le^{x/a} epitopes. Thus, the synthesis of sialyl Le^{x/a} is a suitable example of an embodiment of the method of the invention.

As used herein, “GBSIa” refers to type Ia group B Streptococcus; “SLe” refers to sialyl Lewis antigen; “NeuAc” refers to N-Acetyl neuraminic acid; “Gal” refers to galactopyranose,; “Glc” refers to glucopyranose; and, “GlcNAc” refers to N-Acetyl glucopyranos-amine.

### EXAMPLE 1

Isolation of GBSIa Capsular Polysaccharide

GBSIa capsular polysaccharide was isolated substantially as described in WO 9932653 (Michon, Blake). Briefly, wet GBSIa killed with formaldehyde (ca. 450 g)was suspended in 0.2 N NaOH (1 L), and the mixture was gently stirred overnight. The insoluble materials were removed through centrifugation (7000 rpm, 2 h). The alkaline solution was dialyzed against tap water for 2 days and lyophilized. A solution of the above with insoluble materials in 0.01 M PBS (pH 7.3, 200 mL) was extracted with 90% phenol (“PhOH”) (200 mL). The aqueous phase (upper) was dialyzed and lyophilized to give an amorphous (5-6 g). To a solution of above in water (200 mL) was added proteinase (50 mg). After 16 h at 37° C. the mixture was dialyzed and lyophilized to give an amorphous (ca. 1.6 g). 1 H NMR indicates neither protein nor nucleic acid was left but there was significant amount of group antigens (rhamannans). The above material was treated with 0.1 N NaOH at 90-100° C. for 6 h to break down rhamannans. Upon cooling the mixture was neutralized with acetic anhydride (re-N-acetylation), dialyzed against water overnight and lyophilized to afford a mass (c.a. 1.0 g). Final purification was performed on a Biogel A 0.5 column with 0.01 M PBS (pH 7.3) as eluent. The fractions were pooled, dialyzed, and lyophilized to yield pure polysaccharide (ca. 400 mg).

Products designated “a” and “b” have the structures shown in FIGS. 3-8 with “R” defined as indicated in those figures. In contrast, GBSIa and GBSIb differ in the linking position of galactose and N-Ac-glucosamine (β1,4 and β1,3, respectively), as shown in FIGS. 1 and 2.

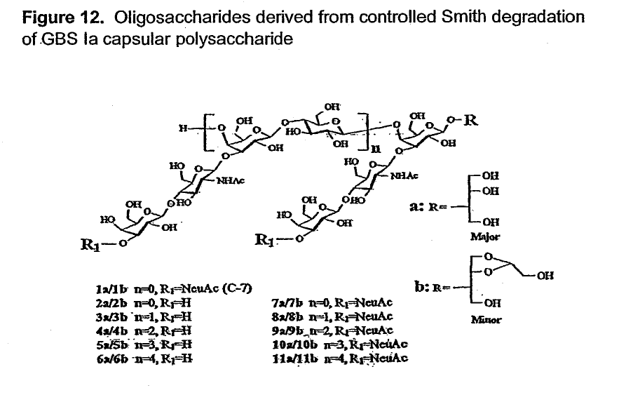

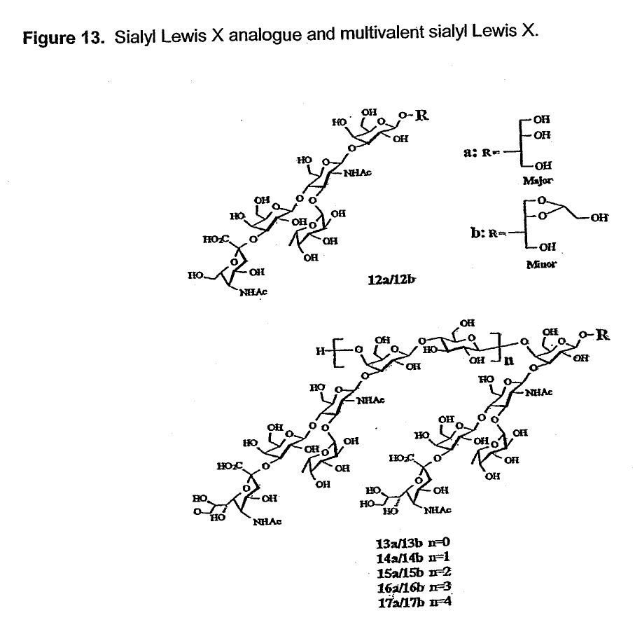

Reaction products and intermediates identified by number and letter are depicted in FIGS. 12 and 13.

### EXAMPLE 2

Smith Degradation of GBSIa Polysaccharide

Part A: Tetrasaccharides (1a/1b) and core trisaccharides (2a/2b)

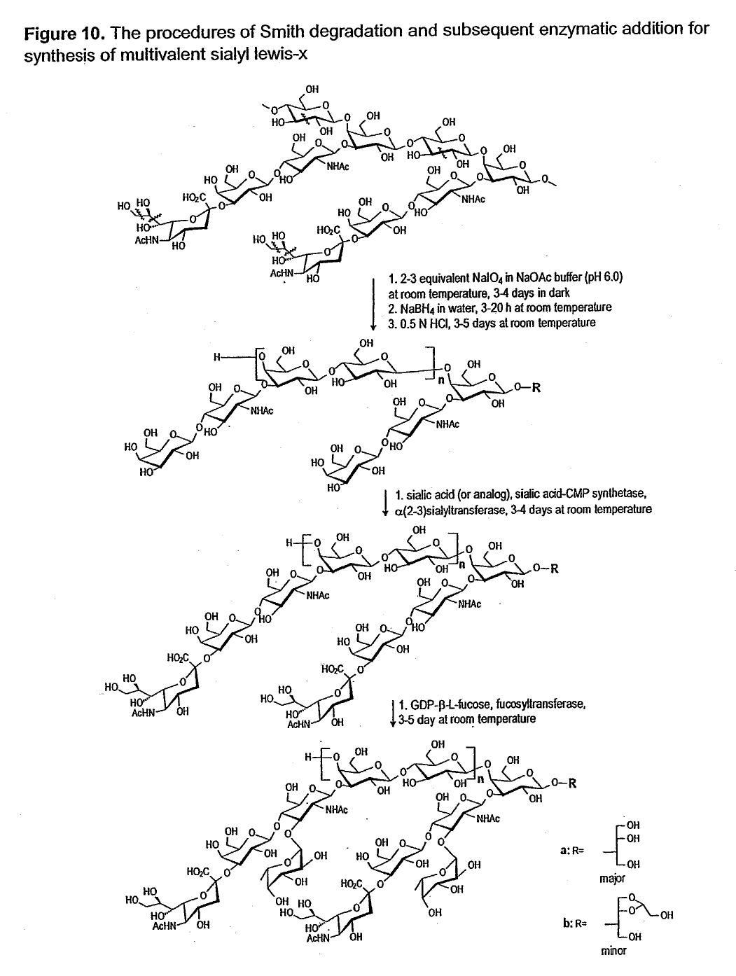

A schematic representation of the Smith degradation is provided in the first portion of FIG. 10 and a summary figure depicting reaction products from Examples 2-4 is provided in FIG. 12.

In general, Smith degradation employs sodium periodate to oxidize (primarily) vicinol diols in a carbohydrate to aldehydes, which are reduced by any suitable reagent, commonly sodium borohydride. To a solution of GBSIa polysaccharide (20 mg, c.a. 0.02 mmole) in 0.2 M NaOAc buffer (pH 6.0, 2 mL) was added 0.1 M NaIO₄ (2 mL, 0.2 mmole). After three days in the dark the solution was dialyzed for two days and then treated with NaBH₄ (16 mg) overnight, dialyzed again and lyophilized to a white powder (15 mg). The above material was treated with 0.5 N HCl (2 mL) at 4° C. for 16 h, neutralized with 1 N NaOH and lyophilized to a powder. The final separation was performed on a Biogel P-6 column using 0.03 M NH₄HCO₃ as eluent to give 1a/1b (7 mg) as major product and 2a/2b (4 mg) as minor product.

Part B: Oligosaccharides 3a/3b through 6a/6b

To a solution of GBSIa polysaccharide (100 mg, 0.1 mmole) in 0.1 M NaOAc (pH 6.0) was added 0.5 M NaIO₄ (0.6 mL, 0.3 mmole). Following the same procedure as described above a white powder (72 mg) was obtained after NaBH₄ (10 mg) reduction. The above material was treated with 0.5 N HCl (10 mL) at room temperature for 4 days when ¹NMR showed completion of sialic acid hydrolysis. The solution was passed through a Dowex 1×8 (HCO3-) column with water as eluent to remove sialic acid and HCl, and lyophilized to a powder. The final separation was performed on a Biogel P-6 column using 0.03 M NH₄HCO₃ as eluent. Fractions were collected and lyophilized to afford pure 2a/2b (9-18 mg), 3a/3b (6-13 mg) and 4a/4b (2-4 mg), a mixture of 5a/5b and 6a/6b (4 mg), and other higher oligomers.

The structure of GBSIa polysaccharide is shown in FIG. 1. GBSIa polysaccharide was treated with sodium perorate (between 2.0-3.0 equivalent) in 0.1 M sodium acetate buffer at pH 6.0. The oxidation degree of 2,3-diol on backbone Glc residues was estimated by ¹H NMR spectroscopy according to the integration of H-2 of intact Glc residues at 3.2 ppm. Reduction of aldehydes with sodium borohydride followed by acid hydrolysis with 0.5 M HCl at 4° C. for 16 h generated a mixture of oligomers. Most sialic acid (NeuAc-7) linkages survived the acid hydrolysis. Partial removal of sialic acid in the multi-repeating units can sometimes occur, making separation of these oligomers difficult. In such situations, complete acid hydrolysis may be preferable.

As an alternative the complete acid hydrolysis was performed under 0.5 M HCl at room temperature for 4 days. Because free sialic acid exists only in β-isomer, the hydrolysis of sialic acid was monitored by ¹H NMR with the disappearance of a resonance at 2.65 ppm for α(2-3) -linked NeuAc and appearance at 2.30 ppm for H-3e of free sialic acid. After removal of free sialic acid through a Dowex 1×8 column the core oligomers were eluted through a Bio-gel P-6 column with 0.03 M NH₄HCO₃ to afford 2a/2b through 6a/6b (see FIG. 12) which represent up to five repeating units of the core structure of GBSIa polysaccharide.

During Smith degradation acid-catalysed cleavage of gylcosidic linkages was undertaken in two different ways based on the products obtained. Complete hydrolysis of glycosidic acetal gave D-threitol as an aglycan (1-6a), whereas, formation of a more stable acetal, 3,4-di-O-hydroxyethylidene-D-threitol (1-6b) (as illustrated in FIG. 12), was also possible after cleavage of glycosidic acetal. The two forms of oligomers, a and b, were not separable under gel-permeation chromatography, but were well characterized by ES-MS and mass spectroscopy-mass spectroscopy (“MS-MS”) analysis. The presence of (═CHCH₂OH) in 1-6b was evidenced by the observation of a mass difference of 42 throughout MS analysis. The ratio of a to b was estimated about 3:1 based on the molecular peak height in MS spectroscopy.

### EXAMPLE 3

Sialylation of Products of Smith Degradation of GBSIa

A solution of 2a/2b, 3a/3b, 4a/4b or a mixture of 5a/5b-6a/6b (2 mg each), NeuAc (4 mg), CTP (4 mg) in water (1 mL) were added 0.1 M MgCl₂ (0.05 ml) and 0.1M MnCl₂ (10 uL), and adjusted to pH 7.5 by the addition of sodium cacodylate. To above solution were added alkaline phosphatase (2 U), NeuAc-CMP synthetase (0.5 mU) and (2-3)sialyltransferase (0.1 U), respectively. Again the pH was adjusted to 7.5 and the mixture was incubated for 3 days at room temperature. Purification on a Biogel P-6 column, using 0.03 M NH₄HCO₃ as eluent, afforded oligosaccharides 7a/7b, 8a/8b, and 9a/9b (c.a. 2.0 mg each), respectively. Products from 5a/5b and 6a/6b were passed through a Superdex 30 column, with 0.33 mM PBS with 5 mM NaCl (pH 7.1), without successful separation, to afford a mixture of 10a/10b and 11a/11b.

Sialylation on 2a/2b through 6a/6b was performed using a combination of NeuAc-CMP synthetase and α(2-3)sialyltransferase to furnish 7a/7b through 11a/11b, respectively, which represent the repeating units of GBSIa polysaccharide. One, two and three repeating units (7a/7b, 8a/8b, and 9a/9b) were obtained as pure forms after purification on a Biogel P-6 column, whereas higher oligomers were obtained as a mixture. Neither Biogel P-10 nor Superdex 30 columns was able to separate these oligomers under the conditions examined.

GBSIa polysaccharide exhibits a conformational epitope that is length-dependent, with which the immune system selects to induce protective antibodies to avoid the problem of inducing antibodies that cross-react with self-antigens. These GBSIa oligosaccharides repeating units are very useful probes to define the GBSI conformational epitope and the factors governing the conformational epitope and its antigenicity/immunogenicity.

### EXAMPLE 4

Fucosylation of Products of Smith Degradation of GBSIa

Reaction products identified by number and letter are depicted in FIG. 13.

To a solution of oligosaccharides 1a/1b, 7a/7b, 8a/8b, 9a/9b (from Examples 2 and 3) and a mixture of 10a/10b-11a/11b (2.0 mg) (from Example 3) and Guanosine-5′-diphosphate-β-L-fucose (“GDP-β-L-Fuc”) (2.0 mg) in HEPES-NaOH buffer (0.5 mL, pH 7.5, 50 mM, 20 mM MnCl₂) was added α(1-3)fucosyltransferase Vl (10 mU, 5μL, CalBiochem, La Jolla, Calif.). More GDP-β-L-Fuc was added after 24 h, and the mixture was kept at 37° C. for 5 days. The mixture was passed through a Biogel P-6 column, using 0.03 M NH₄HCO₃ as eluent, to afford oligosaccharides 12a/12b, 13a/13b, 14a/14b, 15a/15b, and a mixture of 16a/16b and 17a/17b (2.0 mg), respectively.

The fucosylation of 1a/1b gave 12a/12b, a mixture of sialyl Le^{x} analogues in which a C9-C8 chain of NeuAc was truncated. These oligosaccharides are potentially valuable in biological studies because modified NeuAc is a poor substrate for various neurominidases, therefore, these analogues may be more stable and long lasting in vivo.

Similar fucosylations were also performed on 7a/7b through 11a/11b. Oligosaccharides carrying single (13a/13b) or multiple sialyl Le^{x} epitopes (14a/14b through 17a/17b) were obtained, respectively. In general, fucosylation proceeded faster in smaller oligosaccharides in the mixture of multi-repeating units than in larger oligosaccharides. The rate fucosylation of small GBSIa oligosaccharide repeating units was generally higher than that of larger polysaccharides although fucosylation of native polysaccharides was observed.

### EXAMPLE 5

Preparation of Multivalent Le^{x} Antigens

To a solution of 3a/3b, 4a/4b or a mixture of 5a/5b-6a/6b (2 mg each), and GDP-β-L-Fuc (2.0 mg) in HEPES-NaOH buffer (0.5 mL, pH 7.5, 50 mM, 20 mM MnCl₂) are added α(1-3)fucosyltransferase Vl (10 mU, 5 μL, CalBiochem, La Jolla, Calif.). More GDP-β-L-Fuc is added after 24 h, and the mixture is kept at 37° C. for 5 days. Routine purification affords oligosaccharides representing multivalent Le^{x} antigens, 41a/b, 42a/b, 43a/b, 44a/b, respectively.

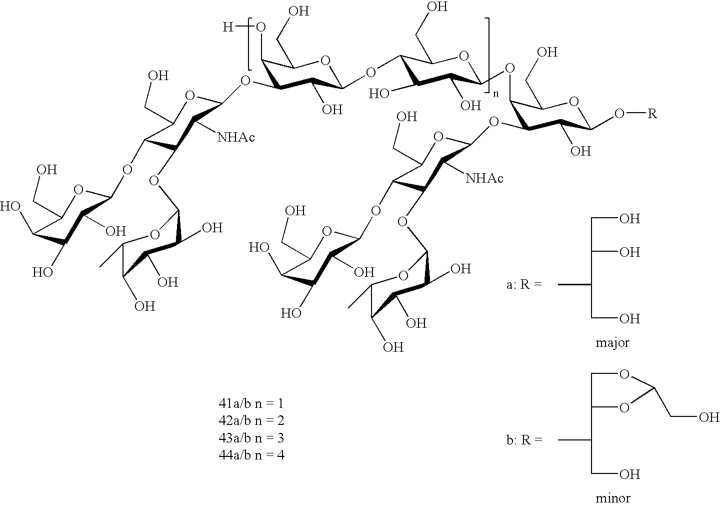

### EXAMPLE 6

Smith Degradation of GBSIb Polysaccharide

To a solution of GBSIb polysaccharide (100 mg, 0.1 mmole) in 0.1 M NaOAc (pH 6.0) were added 0.5 M NaIO₄ (0.6 mL, 0.3 mmole). Following the same procedure as described above a white powder (72 mg) was obtained after NaBH₄ (10 mg) reduction. The above material was treated with 0.5 N HCl (10 mL) at room temperature for 4 days when ¹NMR showed completion of sialic acid hydrolysis. The solution was passed through a Dowex 1×8 (HCO3-) column with water as eluent to remove sialic acid and HCl, and lyophilized to a powder. The final separation was performed on a Biogel P-6 column using 0.03 M NH₄HCO₃ as eluent. Fractions were collected and lyophilized to afford pure oligomers, 51a/b, 52a/b, 53a/b, 54a/b (3-10 mg each), and other higher oligomers.

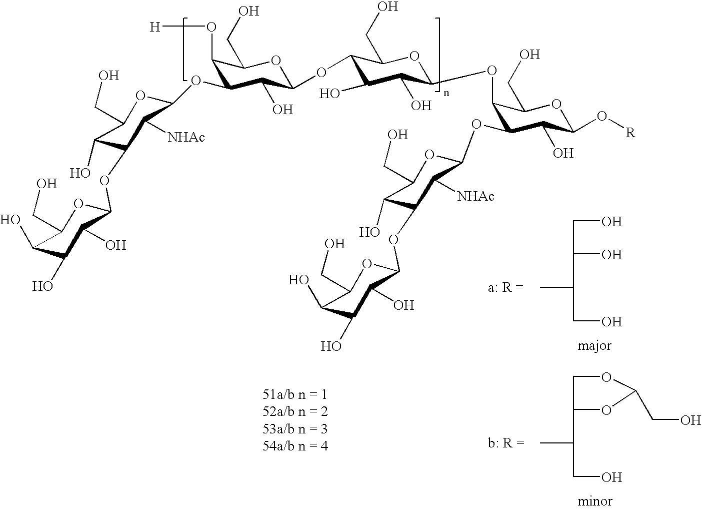

### EXAMPLE 7

Sialylation of Products of Smith Degradation of GBSIb

A solution of 51a/b, 52a/b, 53a/b, and 54a/b (2 mg each) (as obtained in Example 6), NeuAc (4 mg), CTP (4 mg) in water (1 mL) were added 0.1 M MgCl₂ (0.05 ml) and 0.1 M MnCl₂ (10 uL), and adjusted to pH 7.5 by the addition of sodium cacodylate. To above solution were added alkaline phosphatase (2 U), NeuAc-CMP synthetase (0.5 mU) and (2-3)sialyltransferase (0.1 U), respectively. Again the pH was adjusted to 7.5 and the mixture was incubated for 3 days at room temperature. Purification on a Biogel P-6 column, using 0.03 M NH₄HCO₃ as eluent, afforded GBSIb oligosaccharides 55a/b, 56a/b, 57a/b, and 58a/b (c.a. 2.0 mg each), respectively.

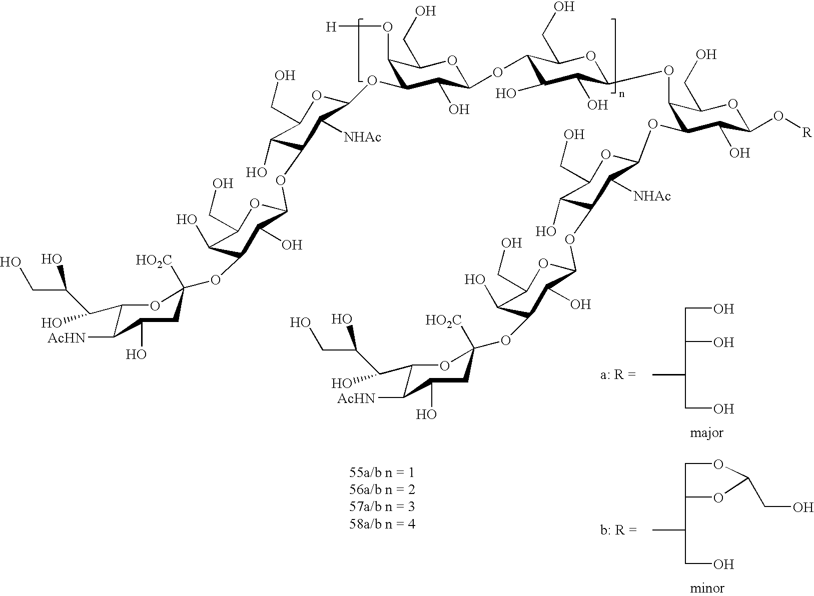

### EXAMPLE 8

Preparation of Multivalent Le^{a} and Sialyl Le^{a} from Products of Smith Degradation of GBSIb

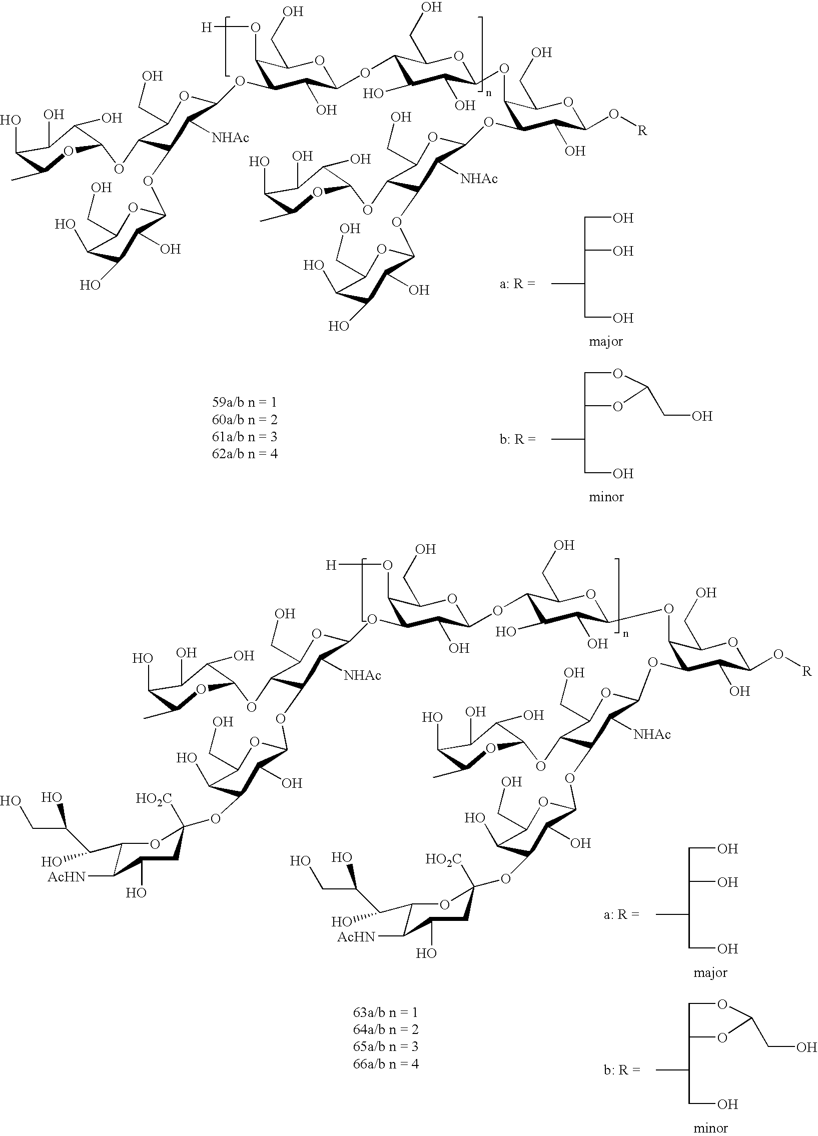

To a solution of oligosaccahrides, 51a/b, 52a/b, 53a/b, 54a/b (2.0 mg) (as obtained in Example 6) and GDP-β-L-Fuc (2.0 mg) in HEPES-NaOH buffer (0.5 mL, pH 7.5, 50 mM, 20 mM MnCl2) are added α(1-3)fucosyltransferase III (10 mU, 5 μL, CalBiochem, La Jolla, Calif.). More GDP-β-L-Fuc is added after 24 h, and the mixture is kept at 37° C. for 5 days. The mixture is passed through a Biogel P-6 column, using 0.03 M NH₄HCO₃ as eluent, to afford oligosaccharides carrying multivalent Le^{a} antigens, 59a/b, 60a/b, 61a/b, 62a/b (2.0 mg), respectively.

Similar fucosylations are also performed on 55a/b, 56a/b, 57a/b, 58a/b. Oligosaccharides carrying multiple sialyl Le^{a} epitopes (63a/b, 64a/b, 65a/b, 66a/b) are obtained, respectively.

### EXAMPLE 9

¹H NMR (D₂O) Examination of Certain Reaction Products

Smith degradation of GBSIa polysaccharide followed by sialylation and/or fucosylation were performed substantially as described in Examples 1 to 4.

Selected reaction products were examined by routine means using ¹H NMR (D₂O). For ease of reference, these reaction products were numbered differently from the reaction products described in Examples 2 to 4, as follows:

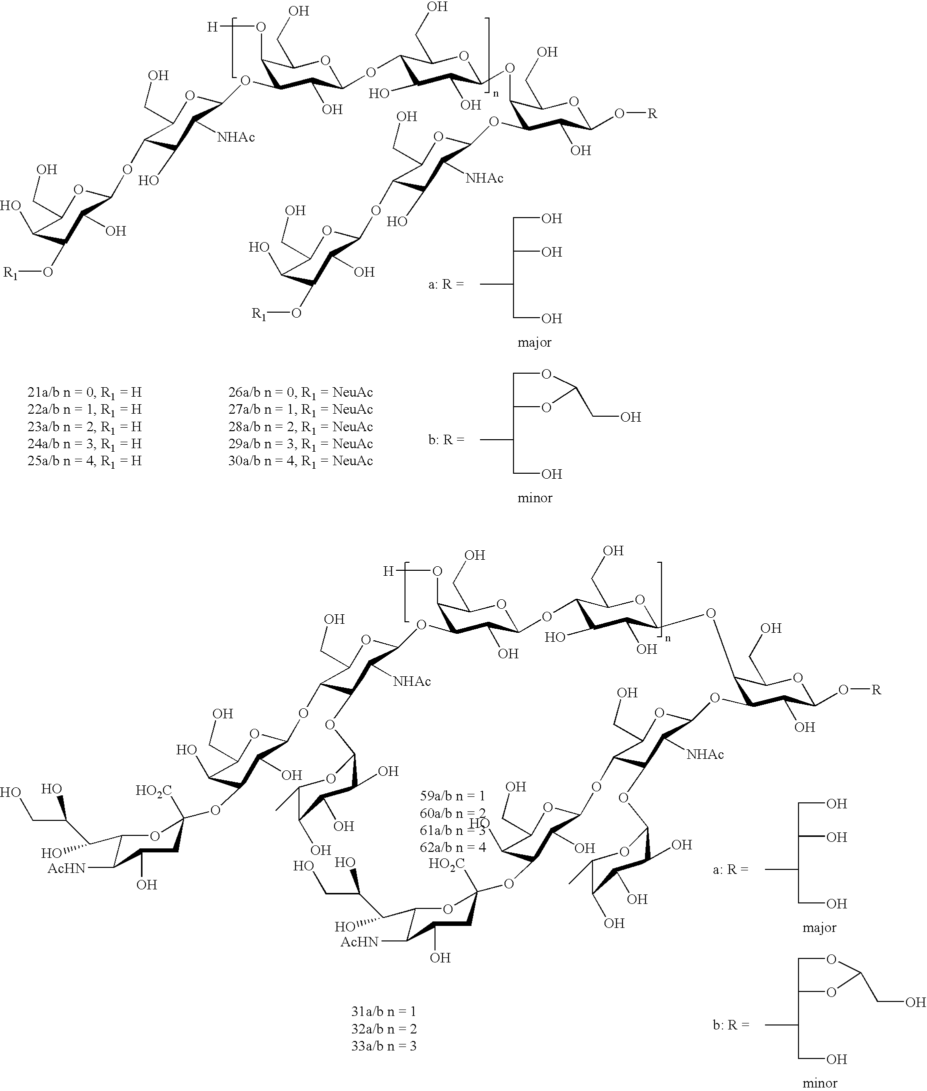

¹H NMR results were obtained as follows:

21a/b δ 2.008 (3 H, s, NHAc), 4.130 (1 H, bs, H-4 of Gal), 4.466 (1 H, d, H-1 of Gal, J_{1,2} 7.5 Hz), 4.482 (1 H, d, H-1 of Gal's J_{1,2} 7.5 Hz), 4.708 (1 H, d, H-1 of GlcNAc, J_{1,2} 8.0 Hz) ppm 22a/b δ 2.001 and 2.011 (6 H, 2s, 2×NHAc), 3.301 (1 H, dd, H-2 of Glc, J_{1,2} 7.5, J_{2,3} 9.0 Hz), 4.129 (1 H, bs, H-4 of Gal), 4.357 (1 H, bs, H-4 of Gal), 4.414 (1 H, d, H-1 of Gal, J_{1,2} 8.0 Hz), 4.455 (2 H, d, 2×H-1 of Gal's, J_{1,2} 8.0 Hz), 4.470 (1 H, d, 2 H-1 of Gal, J_{1,2} 8.0 Hz), 4.683 (1 H, d, H-1 of GlcNAc, J_{1,2} 7.5 Hz ), 4.697 (1 H, d, H-1 of GlcNAc, J_{1,2} 8.5 Hz), 4.904 (1 H, d, H-1 of Glc, J_{1,2} 7.5 Hz) ppm, 23a/b δ 2.030 (9 H, s, 3×NHAc), 3.313 (2 H, bdd, 2×H-2 of Glc), 4.145 (1 H, bs, H-4 of Gal), 4.385 and 4.400 (1 H each, 2×bs, 2×H4 og Gal), 4.416 (1 H, d, H-1 of Gal, J_{1,2} 8.0 Hz), 4.435 (1 H, d, H-1 of Gal, J_{1,2} 8.0 Hz), 4.456 (3 H, d, 3×H-1 of Gal's, J_{1,2} 8.0 Hz), 4.496 (1 H, d, H-1 of Gal, J_{1,2} 8.0 Hz), 4.710 (3 H, bd, 3×H-1 of GlcNAc, J_{1,2} 7.5 Hz), 4.910 (2 H, d, 2×H-1 of Glc, J_{1,2} 8.0 Hz) ppm. ESIMS: 21a/b Calcd for C₂₄H₄₃NO₁₉/C₂₆H₄₅NO₂₀ (649.60/691.64); Found 650.3/692.3 (M+1) and 672.2/714.3 (M+Na); 22a/b calcd for C₅₀H₈₆N₂O₃₉/C₅₂H₈₈N₂O₄₀ (1339.22/1381.26); Found 1338.5/1381.8; 23a/b Calcd for C₇₆H₁₂₉N₃O₅₉/C₇₈H₁₃₁N₆O₆₀ (2028.84/2070.88); Found 2028.1/2071.2.

26a/b δ 1.771 (1 H, dd, H-3a of NeuAc, J_{3a,4}=J_{3e,3a} 11.5 Hz), 2.003 (6 H, s, 2×NHAc), 2.730 (1 H, dd, H-3e of NeuAc, J_{3e,4} 4.0 Hz), 4.469 (1 H, d, H-1 of Gal, J_{1,2} 7.5 Hz), 4.557 (1 H, d, H-1 of Gal's, J_{1,2} 7.5 Hz), 4.705 (1 H, d, H-1 of GlcNAc, J_{1,2} 8.0 Hz) ppm; 27a/b δ 1.775 (2 H, dd, 2×H-3a of NeuAc, J_{3a,4}=J_{3e,3a} 11.5 Hz), 2.005 (12 H, s, 4×NHAc), 2.732 (2 H, dd, 2×H-3e of NeuAc, J_{3e,4} 4.0 Hz), 3.293 (1 H, dd, H-2 of Glc, J_{1,2} 7.5, J_{2,3} 9.0 Hz), 4.410 (1 H, d, H-1 of Gal, J_{1,2} 8.0 Hz), 4.469 (1 H, d, H-1 of Gal, J_{1,2} 8.0 Hz), 4.533 (2 H, d, 2×H-1 of Gal's, J_{1,2} 8.0 Hz), 4.684 (1 H, d, H-1 of GlcNAc, J_{1,2} 7.5 Hz), 4.691 (1 H, d, H-1 of GlcNAc, J_{1,2} 8.5 Hz), 4.890 (1 H, d, H-1 of Glc, J_{1,2} 7.5 Hz) ppm; 28a/b (22° C.) δ 1.800 (3 H, dd, 3×H-3a of NeuAc, J_{3a,4}=J_{3e,3a} 11.5 Hz), 2.030 (18 H, s, 6×NHAc), 2.756 (3 H, dd, 3×H-3e of NeuAc, J_{3e,4} 4.0 Hz), 3.315 (2 H, bdd, 2×H-2 of Glc), 4.115 (3 H, dd, 3×H-3 of Gal's, J_{2,3} 9.5, J_{3,4} 3.0 Hz), 4.154 (H, bs, H-4 of Gal), 4.386 and 4.401 (1 H each, 2×bs, 2×H-4 of Gal), 4.441 (1 H, d, H-1 of Gal, J_{1,2} 8.0 Hz), 4.457 (1 H, d, H-1 of Gal, J_{1,2} 8.0 Hz), 4.496 (1 H, d, H-1 of Gal, J_{1,2} 8.0 Hz), 4.558 (3 H, d, 3×H-1 of Gal's, J_{1,2} 8.0Hz), 4.708 (3 H, bd, 3×H-1 of Glc-NAc, J_{1,2} 7.5 Hz), 4.916 (2 H, d, 2×H-1 of Glc, J_{1,2} 8.0 Hz) ppm. ESIMS: 26a/b Calcd for C₃₅H₆₀N₂O₂₇/C₃₇H₆₂N₂O₂₈ (940.85/982.89); Found 941.0/982.0; 27a/b Calcd for C₇₂H₁₂₀N₄O₅₅/C₇₄H₁₂₂N₄O₅₆ (1921.73/1963.77); Found 1922.0/1964.0; 28a/b Calcd for C₁₀₉H₁₈₀N₆O₈₃/C₁₁₁H₁₈₂N₆O₈₄ (2902.61/2944.65); Found 2901.6/2943.6; 29a/b and 30a/b Calcd for C₁₄₆H₂₄₀N₈O₁₁₁/C₁₄₈H₂₄₂N₈O₁₁₃ (3883.49/3925.53) and C₁₈₃H₃₀₀N₁₀O₁₃₉/C₁₈₅H₃₀₂N₁₀O₁₄₀ (4864.37/4906.41); Found 3882.0/3924.0 and 4863.0/4905.0.

31a/b (15° C.) δ 1.151 (6 H, d, 2×6-CH₃ of Fuc, J_{5,6} 6.5 Hz), 1.782 (2 H, dd, 2×H-3a of NeuAc, J_{3a,4}=J_{3e,3a} 12.0 Hz), 1.994 and 2.013 (3 H and 9 H, 2s, 4×NHAc), 2.747 (2 H, dd, 2×H-3e of NeuAc, J_{3e,4} 3.5 Hz), 3.291 (1 H, dd, H-2 of Glc, J_{1,2} 8.0, J_{2,3} 9.0 Hz), 4.073 (2 H, bd, 2×H-3 of Gal's, J_{2,3} 9.5 Hz), 4.142 (1 H, d, H-4 of Gal), 4.376 (1 H, bs, H-4 of Gal), 4.412 (1 H, d, H-1 of Gal, J_{1,2} 8.0 Hz), 4.471 (1 H, d, H-1 of Gal, J_{1,2} 7.5 Hz), 4.520 (2 H, d, H-1 of Gal's, J_{1,2} 7.5 Hz), 4.690 (2 H, d, 2×H-1 of GlcNAc, J_{1,2} 8.0 Hz), 4.820 (2 H, q, 2×H-5 of Fuc, J_{5,6} 6.5 Hz), 4.921 (1 H, d, H-1 of Glc, J_{1,2} 7.5 Hz), 5.108 (2 H, d, 2×H-1 of Fuc, J_{1,2} 3.5 Hz) ppm; 32a/b (40° C.) δ 1.169 (9 H, d, 3×6-CH₃ of Fuc, J_{5,6} 6.5 Hz), 1.787 (3 H, dd, 3×H-3a of NeuAc, J_{3a,4}=J_{3e,3a} 12.0 Hz), 2.016 and 2.033 (3 H and 15 H, 2s, 6×NHAc), 2.766 (3 H, dd, 3×H-3e of NeuAc, J_{3e,4} 3.5 Hz), 3.305 (2 H, bdd, 2×H-2 of Glc), 4.079 (3 H, bd, 3×H-3 of Gal's, J_{2,3} 10.5 Hz), 4.146 (1 H, d, H-4 of Gal), 4.371 and 4.383 (2 H, bs, 2×H-4 of Gal), 4.439 (1 H, d, H-1 of Gal, J_{1,2} 8.0 Hz), 4.471 (1 H, d, H-1 of Gal, J_{1,2} 7.5 Hz), 4.496 (1 H, d, H-1 of Gal, J_{1,2} 8.0 Hz), 4.519 (3 H, d, 3×H-1 of Gal's, J_{1,2} 8.5 Hz), 4.708 (3 H, m, 3×H-1 of GlcNAc), 4.805 (3 H, q, 3×H-5 of Fuc, J_{5,6} 6.5 Hz), 4.911 (2 H, d, 2×H-1 of Glc, J_{1,2} 7.5 Hz), 5.117 (3 H, d, 3×H-1 of Fuc, J_{1,2} 3.5 Hz) ppm. ESIMS: 31a/b Calcd for C₈₄H₁₄₀N₄O₆₃/C₈₆H₁₄₂N₄O₆₄ (2214.02/2256.06); Found 2214.0/2256.0; 32a/b Calcd for C₁₂₇H₂₁₀N₆O₉₅/C₁₂₉H₂₁₂N₆O₉₆ (3341.04/3383.08); Found 3340.9/3383.9; 33a/b Calcd for C₁₇₀H₂₈₀N₈O₁₂₇/C₁₇₂H₂₈₂N₈O₁₂₈ (4468.07/4510.10); Found 4467.0/4509.0.

Thus, it will be apparent that there has been provided an improved method for the synthesis of complex carbohydrates.

1. A method of synthesizing complex carbohydrates comprising:
(a) subjecting a polysaccharide to degradation to produce a shorter product having at least 4 saccharides; and
(b) subjecting said shorter product to an enzyme-mediated process wherein a first sugar moiety and a second sugar moiety are linked to a first and a second site respectively on the shorter product by an O-glycosidic bond to produce multiple potential antigenic epitopes.

2. The method of claim 1 wherein the sugar moieties linked to the shorter product are independently selected from the group consisting of: sialic acid, N-acylated sialic acid, L-fucose, D-galactose, L-galactose, N-acetyl-D-glucosamine, and N-acetyl-D-galactosamine.

3. The method of claim 2 wherein the sugar moieties linked to the shorter product are independently selected from the group consisting of: N-propionated sialic acid, N-butyrated sialic acid and N-benzoylated sialic acid.

4. The method of claim 1 wherein degradation is conducted by either ozonolysis or oxidation-reduction treatment.

5. The method of claim 1 wherein degradation is conducted by Smith degradation.

6. The method of claim 1 including a further step (c) wherein a further sugar moiety is linked to the first or second sugar moiety.

7. The method of claim 1 including an additional step of using a glycosidase to remove a side chain sugar residue prior to commencing step (b).

8. The method of claim 1 wherein said first sugar moiety is linked by a 1,4-O-glycosidic bond.

9. The method of claim 1 wherein said first sugar moiety is linked by a 1,3-O-glycosidic bond.

10. The method of claim 1 wherein said first sugar moiety is linked by a 1,6-O-glycosidic bond.

11. A complex carbohydrate produced by the method of claim 1.

12. The complex carbohydrate of claim 11 which is a bivalent antigen.

13. The complex carbohydrate of claim 11 which is a trivalent antigen.

14. The complex carbohydrate of claim 11 selected from the group consisting of: multivalent Lewis-x antigen, multivalent sialyl Lewis-x antigen, multivalent Lewis-y antigen, multivalent sialyl Lewis-y antigen, multivalent Lewis-a antigen, multivalent sialyl Lewis-a antigen, multivalent Lewis-b antigen, multivalent sialyl Lewis-b antigen.

15. A kit comprising:
(a) reagents suitable for degradation of a polysaccharide to produce a shorter product; and
(b) an enzyme suitable for use in adding a first and a second sugar moiety to different sites on said shorter product by an O-glycosidic bond.

16. The kit of claim 15 further including instructions for carrying out the method of claim 1.

17. Oligosaccharides 3a, 3b, 4a, 4b, 5a, 5b, 6a, 6b, 8a, 8b, 9a, 9b, 10a, 10b, 11a and 11b.

18. Oligosaccharides 14a, 14b, 15a, 15b, 16a, 16b, 17a and 17b.

19. Oligosaccharides 1a, 1b, 2a, 2b, 3a, 3b, 4a, 4b, 5a, 5b, 6a, 6b, 7a, 7b, 8a, 8b, 9a, 9b, 10a, 10b, 11a, 11b, 12a, 12b, 13a, 13b, 14a, 14b, 15a, 15b, 16a, 16b, 17a or 17b produced by the method of claim 1.

20. Oligosaccharides 51a, 51b, 52a, 52b, 53a, 53b, 54a, 54b, 55a, 55b, 56a, 56b, 57a, 57b, 58a and 58b.

21. Oligosaccharides of claim 20 produced according to the method of claim 1.

22. Sialyl Lewis antigen analogues in which C9-C8 of NeuAc is truncated.

23. Analogues of claim 22 wherein the Lewis antigen is selected from the group consisting of sialyl Lewis-x, -a, -b and -y.

24. Analogues of claim 22 wherein the Lewis antigen is Lewis-x.

25. Sialyl Lewis antigen analogues in which the sialic acid residue is N-acylated.

26. A method for the synthesis of GBSIa-derived oligosaccharides and multivalent Le^{x} antigens by a tailor-assembly approach.
